(19) United States
(12) Patent Application Publication
(10) Pub. No.: US 2008/0311564 A1
(43) Pub. Date: Dec. 18, 2008

(54) Sequences and Methods for Detection of Cytomegalovirus

(76) Inventor: Thomas L. Fort, Hanover, PA (US)

Correspondence Address: David W. Highet, VP & Chief IP Counsel;Becton, Dickinson and Company, 1 Becton Drive, MC 110, Franklin Lakes, NJ, 07417-1880, US

(21) Appl. No.: 11/573,119

(22) PCT Filed: Aug. 5, 2005
(86) PCT No.: PCT/US2005/027865

§ 371 Date: Feb. 21, 2008

Related U.S. Application Data
(60) Provisional application No. 60/599,053, filed on Aug. 6, 2004.

(51) Int. Cl. C12Q 1/68; C07H 21/04

(52) U.S. Cl. 435/6; 536/24.3

(57) ABSTRACT

Amplification primers and methods for specific amplification and detection of a CMV target are disclosed. The primer-target binding sequences are useful for amplification and detection of the CMV target in a variety of amplification and detection reactions.

This application claims priority to U.S. Provisional Application Ser. No. 60/599,053, filed Aug. 6, 2004, which is incorporated herein by reference in its entirety.

### TECHNICAL FIELD

The present invention relates to methods of detecting the presence of cytomegalovirus in a clinical sample. The method involves the use of nucleic acid primers to a glycoprotein H gene target.

### BACKGROUND ART

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Cytomegalovirus (CMV) is a member of the herpes virus family, which includes among others herpes simplex virus types 1 and 2, varicella-zoster virus and Epstein-Barr virus. Between 50% and 85% of adults in the United States are infected by this virus by 40 years of age. In general, there are few symptoms and no long-term health consequences for most healthy persons who acquire CMV. Once infected, the virus remains alive, but dormant within the infected individual's body for life. Infectious CMV may be found in the bodily fluids (i.e., urine, saliva, blood, tears, semen, and breast milk) of any previously infected person. Reactivation of the disease rarely occurs unless the individual suffers from a suppressed immune system through the use of therapeutic drugs or disease. CMV remains the most important cause of congenital viral infection in the United States and is an important cause of morbidity and mortality in certain high-risk groups, such as neonates and immunocompromised and immunosuppressed patients. Transmission from mother to infant may occur, causing symptoms that range from moderate enlargement of the liver and spleen (with jaundice) to fatal illness. Although most infected infants will survive with proper treatment, between 80% to 90% will suffer complications early in life such as hearing loss, vision impairment, and varying degrees of mental retardation.

Because the virus causes few symptoms, many CMV infections are never diagnosed. However, infected individuals develop antibodies to the virus that persist in the body for life. Serological tests for CMV are therefore not generally useful for diagnosis of active infection and, while culture methods are currently used, they are slow requiring up to two weeks to obtain a positive result. In addition, human foreskin and embryo lung fibroblasts are the only cells that reproducibly support in vitro replication of CMV. Detection of viral antigenemia is also used to diagnose CMV infection but the technique is somewhat subjective and is laborious when applied to large numbers of specimens. Nucleic acid amplification methods are more sensitive than culture and offer the potential for quicker time-to-results than is possible with either culture or antigen-based detection. Importantly, quantitative nucleic acid amplification methods offer the ability, not only to diagnose active disease, but also to monitor therapeutic efficacy.

A need, therefore, exists for a rapid and sensitive means of detecting CMV in clinical samples.

### DISCLOSURE OF THE INVENTION

The present invention provides an oligonucleotide having a sequence consisting essentially of a target binding sequence of any one of SEQ ID NOs: 1 through 5. In one embodiment, the oligonucleotide consists essentially of the target binding sequence of SEQ ID NOs:2 or 5. In an additional embodiment, the oligonucleotide further comprises a hairpin, G-quartet, restriction site or a sequence which hybridizes to a reporter probe. In a further embodiment, the oligonucleotide is labeled with a detectable label. In one non-limiting embodiment, the label is fluorescent. In yet another embodiment, the oligonucleotide further comprises a sequence required for an amplification or detection reaction. In an additional embodiment, the sequence required for an amplification or detection reaction is a restriction endonuclease recognition site or a DNA polymerase promoter.

The present invention further provides a kit for an amplification or detection reaction comprising an oligonucleotide having a sequence consisting essentially of the target binding sequence of any one of SEQ ID NOs: 1 through 5. In an additional aspect, the kit further comprises one or more bumper primers. In a further aspect, the one or more bumper primers consist essentially of SEQ ID NOs:6, 7, 8, 9, 10 or 11. In another aspect, the kit further comprises a signal primer. In yet another aspect, the kit further comprises a signal primer and a reporter probe, the signal primer consisting essentially of the target binding sequence of SEQ ID NO:12, 13, 14, or 15 and the reporter probe consisting essentially of the target binding sequence of SEQ ID NO:16 or 17. In a further embodiment, the signal primer consists essentially of the target binding sequence of SEQ ID NO:14 and the reporter probe consists essentially of the target binding sequence of SEQ ID NO:16.

The present invention provides a method for detecting the presence or absence of Cytomegalovirus (CMV) in a sample comprising: (a) hybridizing a first primer having a sequence consisting essentially of the target binding sequence of any one of SEQ ID NOs: 1 through 5 to a target sequence and; (b) detecting the hybridized target sequence. In one embodiment, the method further comprises a second primer having a sequence consisting essentially of the target binding sequence of any one of SEQ ID NOs: 1 through 5. In an additional embodiment, the first primer consists essentially of the target binding sequence of SEQ ID NO:2 and the second primer consists essentially of the target binding sequence of SEQ ID NO:5. In a further embodiment, an amplification or detection reaction is used to detect the hybridized target sequence. In an additional non-limiting embodiment, said amplification or detection reaction is selected from the group consisting of Strand Displacement Amplification (SDA), polymerase chain reaction (PCR), transcription mediated amplification (TMA), self sustained sequence replication (SSR), rolling circle amplification or nucleic acid sequence based amplification (NASBA). In yet another embodiment, the method further comprises: (a) combining the sample with a known concentration of CMV internal control nucleic acid; (b) amplifying the target sequence and internal control nucleic acid in an amplification reaction; (c) detecting the amplified target sequence and internal control nucleic acid; and (d) analyzing the relative amounts of amplified target sequence and internal control nucleic acid. In a further embodiment, the first amplification primer further comprises a hairpin, G-quartet, restriction site or a sequence which hybridizes to a reporter probe. In an additional embodiment, the first primer further comprises a restriction endonuclease recognition site or a DNA polymerase promoter.

The present invention provides an oligonucleotide having a sequence consisting essentially of any one of SEQ ID NOs:1 through 5. In one embodiment, the oligonucleotide consists essentially of SEQ ID NOs:2 or 5. In an additional embodiment, the oligonucleotide further comprises a hairpin, G-quartet, restriction site or a sequence which hybridizes to a reporter probe. In a further embodiment, the oligonucleotide is labeled with a detectable label. In one non-limiting embodiment, the label is fluorescent. In yet another embodiment, the oligonucleotide further comprises a sequence required for an amplification or detection reaction. In an additional embodiment, the sequence required for an amplification or detection reaction is a restriction endonuclease recognition site or a DNA polymerase promoter.

The present invention further provides a kit for an amplification or detection reaction comprising an oligonucleotide having a sequence consisting essentially of any one of SEQ ID NOs:1 through 5. In an additional aspect, the kit further comprises one or more bumper primers. In a further aspect, the one or more bumper primers consist essentially of SEQ ID NOs:6, 7, 8, 9, 10 or 11. In another aspect, the kit further comprises a signal primer. In yet another aspect, the kit further comprises a signal primer and a reporter probe, the signal primer consisting essentially of SEQ ID NO:12, 13, 14, or 15 and the reporter probe consisting essentially of SEQ ID NO:16 or 17. In a further embodiment, the signal primer consists essentially of SEQ ID NO:14 and the reporter probe consists essentially of SEQ ID NO:16.

The present invention provides a method for detecting the presence or absence of Cytomegalovirus (CMV) in a sample comprising: (a) hybridizing a first primer having a sequence consisting essentially of any one of SEQ ID NOs: 1 through 5 to a target sequence and; (b) detecting the hybridized target sequence. In one embodiment, the method further comprises a second primer having a sequence consisting essentially of any one of SEQ ID NOs: 1 through 5. In an additional embodiment, the first primer consists essentially of SEQ ID NO:2 and the second primer consists essentially of SEQ ID NO:5. In a further embodiment, an amplification or detection reaction is used to detect the hybridized target sequence. In an additional non-limiting embodiment, said amplification or detection reaction is selected from the group consisting of Strand Displacement Amplification (SDA), polymerase chain reaction (PCR), transcription mediated amplification (TMA), self sustained sequence replication (SSR), rolling circle amplification or nucleic acid sequence based amplification (NASBA). In yet another embodiment, the method further comprises: (a) combining the sample with a known concentration of CMV internal control nucleic acid; (b) amplifying the target sequence and internal control nucleic acid in an amplification reaction; (c) detecting the amplified target sequence and internal control nucleic acid; and (d) analyzing the relative amounts of amplified target sequence and internal control nucleic acid. In a further embodiment, the first amplification primer further comprises a hairpin, G-quartet, restriction site or a sequence which hybridizes to a reporter probe. In an additional embodiment, the first primer further comprises a restriction endonuclease recognition site or a DNA polymerase promoter.

### BRIEF DESCRIPTION OF THE DRAWINGS

FIG. 1 is a schematic illustration of the detection of CMV nucleic acid target sequence in a Strand Displacement Amplification (SDA) reaction according to a method of the invention.

### MODES FOR CARRYING OUT THE INVENTION

The following terms as used herein are defined as follows:

An “amplification primer” is a primer for amplification of a target sequence by extension of the primer after hybridization to the target sequence. Amplification primers are typically about 10-75 nucleotides in length, preferably about 15-50 nucleotides in length. The total length of an amplification primer for Strand Displacement Amplification (SDA) is typically about 25-50 nucleotides. The 3′ end of an SDA amplification primer (the target binding sequence) hybridizes at the 3′ end of the target sequence. The target binding sequence is about 10-25 nucleotides in length and confers hybridization specificity on the amplification primer. The SDA amplification primer further comprises a recognition site for a restriction endonuclease 5′ to the target binding sequence. The recognition site is for a restriction endonuclease that will nick one strand of a DNA duplex when the recognition site is hemimodified, as described for example by G. Walker, et al., Proc. Natl. Acad. Sci. USA 89:392-396 (1992) and G. Walker, et al., Nucl. Acids Res. 20:1691-1696 (1992). The nucleotides 5′ to the restriction endonuclease recognition site (the “tail”) function as a polymerase repriming site when the remainder of the amplification primer is nicked and displaced during SDA. The repriming function of the tail nucleotides sustains the SDA reaction and allows synthesis of multiple amplicons from a single target molecule. The tail is typically about 10-25 nucleotides in length. Its length and sequence are generally not critical and can be routinely selected and modified. As the target binding sequence is the portion of a primer that determines its target-specificity, for amplification methods that do not require specialized sequences at the ends of the target, the amplification primer generally consists essentially of only the target binding sequence. For example, but not by way of limitation, amplification of a target sequence according to the present invention using PCR will employ amplification primers consisting of the target binding sequences of the amplification primers described herein. For amplification methods that require specialized sequences appended to the target other than the nickable restriction endonuclease recognition site and the tail of SDA (e.g., an RNA polymerase promoter for Self Sustained Sequence Replication (3 SR), Nucleic Acid Sequence Based Amplification (NASBA) or Transcription Based Amplification System (TAS)), the required specialized sequence may be linked to the target binding sequence using routine methods for preparation of oligonucleotides without altering the hybridization specificity of the primer.

A “bumper primer” or “external primer” is a primer used to displace primer extension products in isothermal amplification reactions. The bumper primer anneals to a target sequence upstream of the amplification primer such that extension of the bumper primer displaces the downstream amplification primer and its extension product.

The terms “target” or “target sequence” refers to nucleic acid sequences to be amplified. These include the original nucleic acid sequence to be amplified, the complementary second strand of the original nucleic acid sequence to be amplified and either strand of a copy of the original sequence that is produced by the amplification reaction. These copies serve as amplifiable targets by virtue of the fact that they contain copies of the sequence to which the amplification primers hybridize.

Copies of the target sequence that are generated during the amplification reaction are referred to as “amplification products,” “amplimers” or “amplicons.”

The term “extension product” refers to the copy of a target sequence produced by hybridization of a primer and extension of the primer by polymerase using the target sequence as a template.

The term “species-specific” refers to detection, amplification or oligonucleotide hybridization to a species of organism or a group of related species without substantial oligonucleotide hybridization, detection or amplification of DNA from other species of the same genus or species of a different genus.

The term “assay probe” refers to any oligonucleotide used to facilitate detection or identification of a nucleic acid. Detector probes, detector primers, capture probes, signal primers and reporter probes as described below are non-limiting examples of assay probes.

A “signal primer” comprises a 3′ target binding sequence that hybridizes to a complementary sequence in the target and further comprises a 5′ tail sequence that is not complementary to the target (the adapter sequence). Signal primers and methods of their use are described, for example, in U.S. Pat. No. 6,743,582, U.S. Pat. No. 6,656,680 and U.S. Pat. No. 6,316,200, the entire disclosures of which are incorporated herein by reference. The adapter sequence is an indirectly detectable marker selected such that its complementary sequence will hybridize to the 3′ end of the reporter probe described below. The signal primer hybridizes to the target sequence at least partially downstream of the hybridization site of an amplification primer. The signal primer is extended by the polymerase in a manner similar to extension of the amplification primers. Extension of the amplification primer displaces the extension product of the signal primer in a target amplification-dependent manner, producing a single-stranded product comprising a 5′ adapter sequence, a downstream target binding sequence and a 3′ binding sequence specific for hybridization to a flanking SDA amplification primer. Hybridization and extension of this flanking amplification primer and its subsequent nicking and extension creates amplification products containing the complement of the adapter sequence that may be detected as an indication of target amplification. For example, U.S. Pat. No. 6,743,582, U.S. Pat. No. 6,656,680 and U.S. Pat. No. 6,316,200 describe signal primers similar to those outlined above and which are unlabeled. These detection systems utilize a reporter probe (described below) that is fluorescently labeled.

A “reporter probe” according to the present invention functions as a detector oligonucleotide and comprises a label that is preferably at least one donor/quencher dye pair, i.e., a fluorescent donor dye and a quencher for the donor fluorophore. The label is linked to a sequence or structure in the reporter probe (the reporter moiety) that does not hybridize directly to the target sequence. The sequence of the reporter probe 3′ to the reporter moiety is selected to hybridize to the complement of the signal primer adapter sequence. In general, the 3′ end of the reporter probe does not contain sequences with any significant complementarity to the target sequence. If the amplification products containing the complement of the adapter sequence described above are present, they can then hybridize to the 3′ end of the reporter probe. Priming and extension from the 3′ end of the adapter complement sequence allows the formation of the reporter moiety complement. This formation renders the reporter moiety double-stranded, thereby allowing the label of the reporter probe to be detected and indicating the presence of or the amplification of the target.

The term “amplicon” refers to the product of the amplification reaction generated through the extension of either or both of a pair of amplification primers. An amplicon may contain exponentially amplified nucleic acids if both primers utilized hybridize to a target sequence. Alternatively, amplicons may be generated by linear amplification if one of the primers utilized does not hybridize to the target sequence. This term is used generically herein and does not imply the presence of exponentially amplified nucleic acids.

This invention relates to the amplification and detection of nucleic acids from CMV. More specifically, the invention disclosure relates to a specific DNA region found within the glycoprotein H gene of the CMV genome and 17 oligonucleotide probes, which have regions complimentary to the DNA sequence of the CMV glycoprotein H gene. Probes of the specified sequences, or other probes that are complimentary to the specified DNA region, can be used as primers in nucleic acid amplification procedures such as SDA, PCR, or others. These primers, when mixed with other reagents needed for amplification, such as enzymes, deoxynucleotides and buffer components, could be used to amplify nucleic acids from CMV. The probes could also be labeled and used in the direct detection of CMV nucleic acid via hybridization reactions without amplification. The CMV nucleic acid could be found in clinical samples such as urine, saliva, vaginal secretions, blood and plasma.

The present invention provides probes and primers for detection of CMV nucleic acids, which provides a more rapid and sensitive means for detecting CMV than culture-based methods. Further, the probes and primers of the invention may allow for more reliable detection of naturally occurring variants of CMV, as they are based on an analysis of conserved regions of the CMV glycoprotein H gene. The CMV glycoprotein H gene DNA region of interest is 101 base pairs in length. The primers and probes are predicted to facilitate detection and/or quantification of all known strains of CMV. That is, a single amplification primer pair according to the present invention should efficiently amplify all known strains of CMV, which may then be detected in a single detection step using the detector probes and primers of the present invention.

One preferred method involves the use of the disclosed primers and probes in a SDA, tSDA, or homogeneous real-time fluorescent tSDA reaction to detect CMV nucleic acid in a clinical sample for diagnostic purposes. These methods are known to those skilled in the art from references such as U.S. Pat. No. 5,547,861, U.S. Pat. No. 5,648,211, U.S. Pat. No. 5,846,726, U.S. Pat. No. 5,928,869, U.S. Pat. No. 5,958,700, U.S. Pat. No. 5,935,791, U.S. Pat. No. 6,054,279, U.S. Pat. No. 6,316,200, U.S. Pat. No. 6,656,680, U.S. Pat. No. 6,743,582 and U.S. Pat. No. 6,258,546, the disclosures of all of which are hereby specifically incorporated herein by reference. Primers developed for use in SDA are shown in Table 1. Also shown are bumper primers, signal primers and reporter probes for the amplification and detection of the resultant amplicons. The target binding (i.e., CMV-specific) sequences are underlined. The target binding sequence of an amplification primer determines its target specificity.

TABLE 1
Primers for the amplification and detection of CMV DNA

Upstream Primers

| | | |
|---|---|---|
| CMVgpHAL1 | 5′-CGA TTC CGC TCC AGA CTT CTC GGG CGC | SEQ ID NO:1 |
| | GTC AAG AAC TCT | |
| CMVgpHAL3 | 5′-CGA TTC CGC TCC AGA CTT CTC GGG CGC | SEQ ID NO:2 |
| | GTC AAG AAC TCT AC | |

Downstream Primers

| | | |
|---|---|---|
| CMVgpHAR1 | 5′-ACC GCA TCG AAT GCA TGT CTC GGG TCT | SEQ ID NO:3 |
| | CCG TCG TAT GT | |
| CMVgpHAR2 | 5′-ACC GCA TCG AAT GCA TGT CTC GGG CTC | SEQ ID NO:4 |
| | TCC GTC GTA TGT | |
| CMVgpHAR3 | 5′-ACC GCA TCG AAT GCA TGT CTC GGG TCT | SEQ ID NO:5 |
| | CTC CGT CGT ATG T | |

Bumper Primers

| | | |
|---|---|---|
| CMVgpHBL1 | 5′-TTT CTT TCA GCC TTC G | SEQ ID NO:6 |
| CMVgpHBL2 | 5′-TTT TCT TTC AGC CTT C | SEQ ID NO:7 |
| CMVgpHBL3 | 5′-CTT TTC TTT CAG CCT T | SEQ ID NO:8 |
| CMVgpHBR1 | 5′-TGA AGA TTT CGC GTC | SEQ ID NO:9 |
| CMVgpHBR2 | 5′-CGA TGA AGA TTT CGC | SEQ ID NO:10 |
| CMVgpHBR3 | 5′-TAC GAT GAA GAT TTC G | SEQ ID NO:11 |

Signal Primers

| | | |
|---|---|---|
| CMVgpHA1 | 5′-ACG TTA GCC ACC ATA CGG AT TCA TGG | SEQ ID NO:12 |
| | GCA GCC TCG TCC ACT | |
| CMVgpHA2 | 5′-ACG TTA GCC ACC ATA CGG AT TCA TGG | SEQ ID NO:13 |
| | GCA GCC TCG TCC ACT C | |
| CMVgpHA3 | 5′-ACG TTA GCC ACC ATA CGG AT TCA TGG | SEQ ID NO:14 |
| | GCA GCC TCG TCC ACT CC | |
| CMVgpHA6 | 5′-ACG TTA GCC ACC ATA CGG AT CAT GGA | SEQ ID NO:15 |
| | GTG GAC GAG GCT GCC C | |

Reporter Probes

| | | |
|---|---|---|
| MPC2 (F/D) | 5′-(FAM)-TCC CCG AGT-(DABCYL)-ACT GAT | SEQ ID NO:16 |
| | CCG CAC TAA CGA CT | |
| MPC (D/R) | 5′-(DABCYL)-TCC CCG AGT-(ROX)-ACG TTA | SEQ ID NO:17 |
| | GCC ACC ATA CTT GA | |

A DNA-based internal control may also be incorporated in the reaction mixture that co-amplifies with the CMV target sequences of the present invention. The internal control is designed to verify negative results and identify potentially inhibitory samples. Such a control may be used for the purposes of quantification in a competitive DNA assay format similar to that describes for RNA by Nadeau et al., Anal. Biochem. 276:177-187 (1999).

As nucleic acids do not need to be completely complementary in order to hybridize, it is to be understood that the probe and primer sequences disclosed herein may be modified to some extent without loss of utility as CMV-specific probes and primers. Hybridization of complementary and partially complementary nucleic acid sequences may be obtained by adjustment of the hybridization conditions to increase or decrease stringency (i.e., adjustment of hybridization pH, temperature or salt content of the buffer). Such modifications of the disclosed sequences and any necessary adjustments of hybridization conditions to maintain CMV-specificity may be considered minor.

The amplification products generated using the primers disclosed herein may be detected by a characteristic size, for example, but not by way of limitation, on polyacrylamide or agarose gels stained with ethidium bromide. Alternatively, amplified target sequences may be detected by means of an assay probe, which is an oligonucleotide tagged with a detectable label. In one embodiment, at least one tagged assay probe may be used for detection of amplified target sequences by hybridization (a detector probe), by hybridization and extension as described by Walker, et al., Nucl. Acids Res., supra (a detector primer) or by hybridization, extension and conversion to double stranded form as described in EP 0 678 582 (a signal primer).

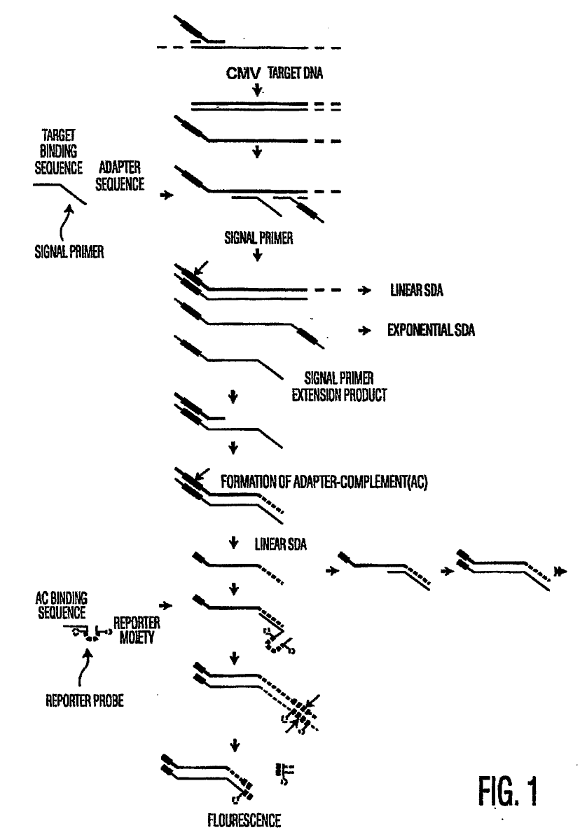

One embodiment for the detection of amplified target according to the present invention is illustrated schematically in FIG. 1. In this embodiment, the 5′ tail sequence of the signal primer is comprised of a sequence that does not hybridize to the target (the adapter sequence). See U.S. Pat. No. 6,743,582, U.S. Pat. No. 6,656,680 and U.S. Pat. No. 6,316,200. The adapter sequence is an indirectly detectable marker that may be selected such that it is the same in a variety of signal primers that have different 3′ target binding sequences (i.e., a “universal” 5′ tail sequence). SEQ ID NOs:12-15 are particularly useful as signal primers, in conjunction with the amplification primers of the invention for detection of CMV. Preferably, an assay probe is a single reporter probe sequence that hybridizes to the adapter sequence complement of the signal primers of the invention. Alternatively, an assay probe can be selected to hybridize to a sequence in the target that is between the amplification primers. In a further embodiment, an amplification primer or the target binding sequence thereof may be used as the assay probe.

The detectable label of the assay probe is a moiety that can be detected either directly or indirectly as an indication of the presence of the target nucleic acid. For direct detection of the label, assay probes may be tagged with a radioisotope and detected by autoradiography or tagged with a fluorescent moiety and detected by fluorescence as is known in the art. Alternatively, the assay probes may be indirectly detected by tagging with a label that requires additional reagents to render it detectable. Indirectly detectable labels include, for example, but not by way of limitation, chemiluminescent agents, enzymes that produce visible reaction products, and ligands (e.g., haptens, antibodies or antigens) that may be detected by binding to labeled specific binding partners (e.g., antibodies or antigens/haptens). Ligands are also useful for immobilizing the ligand-labeled oligonucleotide (the capture probe) on a solid phase to facilitate its detection. Particularly useful labels include biotin (detectable by binding to labeled avidin or streptavidin) and enzymes such as horseradish peroxidase or alkaline phosphatase (detectable by addition of enzyme substrates to produce colored reaction products). Methods for adding such labels to, or including such labels in, oligonucleotides are well-known in the art and any of these methods are suitable for use in the present invention.

Examples of specific detection methods that may be employed include a chemiluminescent method in which amplified products are detected using a biotinylated capture probe and an enzyme-conjugated detector probe as described in U.S. Pat. No. 5,470,723. After hybridization of these two assay probes to different sites in the assay region of the target sequence (between the binding sites of the two amplification primers), the complex is captured on a streptavidin-coated microtiter plate by means of the capture probe, and the chemiluminescent signal is developed and read in a luminometer.

Amplification primers for specific detection and identification of nucleic acids may be packaged in the form of a kit. Typically, such a kit contains at least one pair of amplification primers. The kit may further optionally include an amplification control sequence to be co-amplified with the target sequence. Reagents for performing a nucleic acid amplification reaction such as buffers, additional primers, nucleotide triphosphates, enzymes, etc., may also be included with the target-specific amplification primers. The components of the kit are packaged together in a common container, optionally including instructions for performing a specific embodiment of the inventive methods. Other optional components may also be included in the kit, e.g., an oligonucleotide tagged with a label suitable for use as an assay probe, and/or reagents or means for detecting the label.

The target binding sequences of the amplification primers confer species hybridization specificity on the oligonucleotides and, therefore, provide species specificity to the amplification reaction. The target binding sequences of the amplification primers of the invention are also useful in other nucleic acid amplification protocols such as PCR, conventional SDA (a reaction scheme that is essentially the same as that of tSDA but conducted at lower temperatures using mesophilic enzymes), 3SR, NASBA and TAS. Specifically, any amplification protocol that utilizes cyclic, specific hybridization of primers to the target sequence, extension of the primers using the target sequence as a template and separation or displacement of the extension products from the target sequence may employ the target binding sequences of the present invention. For amplification methods that do not require specialized, non-target binding sequences (e.g., PCR), the amplification primers may consist only of the target binding sequences of the amplification primers listed in Table 1.

Other sequences, as required for performance of a selected amplification reaction, may optionally be added to the target binding sequences disclosed herein without altering the species specificity of the oligonucleotide. By way of example, but not of limitation, the specific amplification primers may contain a recognition site for the restriction endonuclease BsoBI that is nicked during the SDA reaction. It will be apparent to one skilled in the art that other nickable restriction endonuclease recognition sites may be substituted for the BsoBI recognition site including, but not limited to, those recognition sites disclosed in EP 0 684 315. Preferably, the recognition site is for a thermophilic restriction endonuclease so that the amplification reaction may be performed under the conditions of tSDA. Similarly, the tail sequence of the amplification primer (5′ to the restriction endonuclease recognition site) is generally not critical, although the restriction site used for SDA and sequences that will hybridize either to their own target binding sequence or to the other primers should be avoided. Some amplification primers for SDA, therefore, consist of 3′ target binding sequences, a nickable restriction endonuclease recognition site 5′ to the target binding sequence and a tail sequence about 10-25 nucleotides in length 5′ to the restriction endonuclease recognition site. The nickable restriction endonuclease recognition site and the tail sequence are sequences required for the SDA reaction. As described in U.S. Pat. No. 6,379,892, incorporated herein by reference in its entirety, some amplification primers for SDA can consist of target specific sequences both 5′ and 3′ of the restriction enzyme recognition site. An increase in the efficiency of target specific hybridization can be attained with this design. For other amplification reactions (e.g., 3 SR, NASBA and TAS), the amplification primers may consist of the target binding sequence and additional sequences required for the selected amplification reaction (e.g., sequences required for SDA as described above or a promoter recognized by RNA polymerase for 3SR). Adaptation of the target binding sequences of the invention to amplification methods other than SDA is contemplated by the present invention. The target binding sequences of the invention may be readily adapted to CMV-specific target amplification and detection in a variety of amplification reactions. In SDA, the bumper primers are not essential for species specificity, as they function to displace the downstream, species-specific amplification primers. It is required only that the bumper primers hybridize to the target upstream from the amplification primers so that when they are extended they will displace the amplification primer and its extension product. The particular sequence of the bumper primer is, therefore, generally not critical and may be derived from any upstream target sequences that are sufficiently close to the binding site of the amplification primer to allow displacement of the amplification primer extension product upon extension of the bumper primer. Occasional mismatches with the target in the bumper primer sequence or some cross-hybridization with non-target sequences do not generally negatively affect amplification efficiency as long as the bumper primer remains capable of hybridizing to the specific target sequence.

Amplification reactions employing the primers of the invention may incorporate thymine as taught by Walker, et al., Nucl. Acids Res., supra, or they may wholly or partially substitute 2′-deoxyuridine 5′-triphosphate for TTP in the reaction to reduce cross-contamination of subsequent amplification reactions, e.g., as taught in EP 0 624 643. Uridine (dU) is incorporated into amplification products and can be excised by treatment with uracil DNA glycosylase (UDG). These abasic sites render the amplification product unamplifiable in subsequent amplification reactions. UDG may be inactivated by uracil DNA glycosylase inhibitor (UGI) prior to performing the subsequent amplification to prevent excision of dU in newly formed amplification products. Alternatively, UDG may be inactivated by heating or, in tSDA, the elevated temperature of the reaction mixture itself may be used to inactivate the enzyme concurrently with initiation of amplification.

SDA is an isothermal method of nucleic acid amplification in which extension of primers, nicking of a hemimodified restriction endonuclease recognition/cleavage site, displacement of single stranded extension products, annealing of primers to the extension products (or the original target sequence) and subsequent extension of the primers occurs concurrently in the reaction mix. This is in contrast to PCR, in which the steps of the reaction occur in discrete phases or cycles as a result of the temperature cycling characteristics of the reaction. SDA is based upon (1) the ability of a restriction endonuclease to nick the unmodified strand of a hemiphosphorothioate form of its double stranded recognition/cleavage site and (2) the ability of certain polymerases to initiate replication at the nick and displace the downstream non-template strand. After an initial incubation at increased temperature (about 95° C.) to denature double stranded target sequences for annealing of the primers, subsequent polymerization and displacement of newly synthesized strands takes place at a constant temperature. Production of each new copy of the target sequence consists of five steps: (1) binding of amplification primers to an original target sequence or a displaced single-stranded extension product previously polymerized, (2) extension of the primers by a 5′→3′ exonuclease deficient polymerase incorporating an α-thio deoxynucleoside triphosphate (α-thio dNTP), (3) nicking of a hemimodified double-stranded restriction site, (4) dissociation of the restriction enzyme from the nick site, and (5) extension from the 3′ end of the nick by the 5′→3′ exonuclease deficient polymerase with displacement of the downstream newly synthesized strand. Nicking, polymerization and displacement occur concurrently and continuously at a constant temperature because extension from the nick regenerates another nickable restriction site. When a pair of amplification primers is used, each of which hybridizes to one of the two strands of a double-stranded target sequence, amplification is exponential. This is because the sense and antisense strands serve as templates for the opposite primer in subsequent rounds of amplification. When a single amplification primer is used, amplification is linear because only one strand serves as a template for primer extension. Non-limiting examples of restriction endonucleases that nick their double stranded recognition/cleavage sites when an α-thio dNTP is incorporated are HincII, HindII, AvaI, NciI and Fnu4HI. All of these restriction endonucleases and others that display the required nicking activity are suitable for use in conventional SDA. They are, however, relatively thermolabile and lose activity above about 40° C.

Targets for amplification by SDA may be prepared by fragmenting larger nucleic acids by restriction with an endonuclease that does not cut the target sequence. It is generally preferred, however, that target nucleic acids having selected restriction endonuclease recognition/cleavage sites for nicking in the SDA reaction be generated as described by Walker, et al., Nucl. Acids Res., supra, and in U.S. Pat. No. 5,270,184 (specifically incorporated herein by reference). Briefly, if the target sequence is double-stranded, four primers are hybridized to it. Two of the primers (S₁ and S₂) are SDA amplification primers and two (B₁ and B₂) are external or bumper primers. S₁ and S₂ bind to opposite strands of double-stranded nucleic acids flanking the target sequence. B₁ and B₂ bind to the target sequence 5′ (i.e., upstream) of S₁ and S₂, respectively. The exonuclease deficient polymerase is then used to extend all four primers simultaneously in the presence of three deoxynucleoside triphosphates and at least one modified deoxynucleoside triphosphate (e.g., 2′-deoxyadenosine 5′-O-(1-thiotriphosphate), “dATPαS”). The extension products of S₁ and S₂ are thereby displaced from the original target sequence template by extension of B₁ and B₂. The displaced, single-stranded extension products of the amplification primers serve as targets for binding of the opposite amplification and bumper primer (e.g., the extension product of S₁ binds S₂ and B₂). The next iteration of extension and displacement results in two double-stranded nucleic acid fragments with hemimodified restriction endonuclease recognition/cleavage sites at each end. These are suitable substrates for amplification by SDA. As in SDA, the individual steps of the target generation reaction occur concurrently and continuously, generating target sequences with the recognition/cleavage sequences at the ends required for nicking by the restriction enzyme in SDA. As all of the components of the SDA reaction are already present in the target generation reaction, target sequences generated automatically and continuously enter the SDA iteration and are amplified.

To prevent cross-contamination of one SDA reaction by the amplification products of another, dUTP may be incorporated into SDA-amplified DNA in place of dTTP without inhibition of the amplification reaction e.g., as taught by EP 0 624 643. The uracil-modified nucleic acids may then be specifically recognized and inactivated by treatment with uracil DNA glycosylase (UDG). Therefore, if dUTP is incorporated into SDA-amplified DNA in a prior reaction, any subsequent SDA reactions can be treated with UDG prior to amplification of double-stranded targets, and any dU containing DNA from previously amplified reactions will be rendered unamplifiable. The target DNA to be amplified in the subsequent reaction does not contain dU and will not be affected by the UDG treatment. UDG may then be inhibited by treatment with UGI prior to amplification of the target.

Alternatively, UDG may be heat-inactivated. In tSDA, the higher temperature of the reaction itself (≦50° C.) can be used concurrently to inactivate UDG and amplify the target.

SDA requires a polymerase that lacks 5′→3′ exonuclease activity, initiates polymerization at a single-stranded nick in double stranded nucleic acids, and displaces the strand downstream of the nick while generating a new complementary strand using the unnicked strand as a template. The polymerase must extend by adding nucleotides to a free 3′-OH. To optimize the SDA reaction, it is also desirable that the polymerase be highly processive to maximize the length of target sequence that can be amplified. Highly processive polymerases are capable of polymerizing new strands of significant length before dissociating and terminating synthesis of the extension product. Displacement activity in the amplification reaction makes the target available for synthesis of additional copies and generates the single-stranded extension product to which a second amplification primer may hybridize in exponential amplification reactions. Nicking activity of the restriction enzyme perpetuates the reaction and allows subsequent rounds of target amplification to initiate.

tSDA is performed essentially as the conventional SDA described by Walker, et al., Proc. Natl. Acad. Sci. and Walker, et al., Nucl. Acids Res., supra, with substitution of the desired thermostable polymerase and thermostable restriction endonuclease. Of course, the temperature of the reaction will be adjusted to the higher temperature suitable for the substituted enzymes and the HincII restriction endonuclease recognition/cleavage site will be replaced by the appropriate restriction endonuclease recognition/cleavage site for the selected thermostable endonuclease. Also in contrast to Walker, et al., Proc. Natl. Acad. Sci., supra, the practitioner may include the enzymes in the reaction mixture prior to the initial denaturation step if they are sufficiently stable at the denaturation temperature. Preferred restriction endonucleases for use in tSDA are BsrI, BstNI, BsmAI, BslI and BsoBI (New England BioLabs), and BstOI (Promega). The preferred thermophilic polymerases are Bca (Panvera) and Bst (New England Biolabs).

Homogeneous real-time fluorescent tSDA is a modification of tSDA that employs reporter oligonucleotides to produce reduced fluorescence quenching in a target-dependent manner. The reporter oligonucleotides contain a donor/acceptor dye pair linked such that fluorescence quenching occurs in the absence of target. Quenching efficiency is a function of the distance between the donor and acceptor dye pairs. In the presence of the target, unfolding or linearization of an intramolecularly base-paired secondary structure in the reporter oligonucleotide, and/or cleavage of the nucleic acid strands separating the donor and acceptor increases the distance between the dyes and reduces fluorescence quenching. Unfolding of a base-paired secondary structure typically involves intermolecular base-pairing between the sequence of the secondary structure and a complementary strand such that the secondary structure is at least partially disrupted, or it may be fully linearized in the presence of a complementary strand of sufficient length. In one embodiment, a restriction endonuclease recognition site (RERS) is present between the two dyes such that intermolecular base-pairing between the region of DNA separating the two dyes and a complementary strand renders the RERS double-stranded and cleavable by a restriction endonuclease. An alternative embodiment involves the use of linear reporter probes that lack secondary structure. In the case of such probes, the donor and acceptor moieties are separated by a stretch of DNA that includes an RERS. When the reporter probe is rendered double stranded during the course of amplification, the RERS becomes a target for recognition by a restriction enzyme that cleaves the DNA, thereby separating the dyes and generating fluorescence. Cleavage by the restriction endonuclease separates the donor and acceptor dyes onto different nucleic acid fragments, further contributing to decreased quenching. In either embodiment, an associated change in a fluorescence parameter (e.g., an increase in donor fluorescence intensity, a decrease in acceptor fluorescence intensity or a ratio of fluorescence before and after unfolding) is monitored as an indication of the presence of the target sequence. Monitoring a change in donor fluorescence intensity is preferred, as this change is typically larger than the change in acceptor fluorescence intensity. Other fluorescence parameters such as a change in fluorescence lifetime may also be monitored.

Many donor/quencher dye pairs known in the art are useful in the present invention. These include, but not limited to, for example, fluorescein isothiocyanate (FITC)/tetramethylrhodamine isothiocyanate (TRITC), FITC/Texas Red™. (Molecular Probes), FITC/N-hydroxysuccinimidyl 1-pyrenebutyrate (PYB), FITC/eosin isothiocyanate (EITC), N-Docket hydroxysuccinimidyl 1-pyrenesulfonate (PYS)/FITC, FITC/Rhodamine X, FITC/tetramethylrhodamine (TAMRA), and others. The selection of a particular donor/quencher pair is not critical. For energy transfer quenching mechanisms it is only necessary that the emission wavelengths of the donor fluorophore overlap the excitation wavelengths of the quencher, i.e., there must be sufficient spectral overlap between the two dyes to allow efficient energy transfer, charge transfer or fluorescence quenching. P-(dimethyl aminophenylazo) benzoic acid (DABCYL) is a non-fluorescent quencher dye which effectively quenches fluorescence from an adjacent fluorophore, e.g., fluorescein or 5-(2′-aminoethyl) aminonaphthalene (EDANS). Any dye pair which produces fluorescence quenching in the detection probe of the invention can be used in the methods of the invention, regardless of the mechanism by which quenching occurs. Terminal and internal-labeling methods are also known in the art and may be routinely used to link the donor and quencher dyes at their respective sites in the detection probe.

Cleavage of an oligonucleotide refers to breaking the phosphodiester bonds of both strands of a DNA duplex or breaking the phosphodiester bond of single-stranded DNA. This is in contrast to nicking, which refers to breaking the phosphodiester bond of only one of the two strands in a DNA duplex.

A reporter oligonucleotide for homogeneous real-time fluorescent tSDA may be an oligonucleotide that comprises both a single-stranded 5′ or 3′ section that hybridizes to the target sequence (the target binding sequence), as well as an adjacent intramolecularly base-paired secondary structure. One embodiment involves the use of linear reporter oligonucleotides as discussed above. In yet another embodiment, as demonstrated in FIG. 1 (and illustrated in U.S. Pat. No. 6,743,582, U.S. Pat. No. 6,656,680 and U.S. Pat. No. 6,316,200), the detector oligonucleotide is a reporter probe that comprises a single-stranded 5′ or 3′ section that does not hybridize to the target sequence. Rather, the single-stranded 5′ or 3′ section hybridizes to the complement of the signal primer adapter sequence (the adapter-complement binding sequence). A further characteristic of the reporter probe is that this hybridizing section is adjacent to an intramolecularly base-paired secondary structure. The detector oligonucleotides of the present invention further comprise a donor/acceptor dye pair linked to the detector oligonucleotide such that donor fluorescence is quenched when the secondary structure is intramolecularly base-paired and unfolding or linearization of the secondary structure results in a decrease in fluorescence quenching.

The detector oligonucleotide reporter probe can alternatively be linear rather than contain a hairpin structure. In this case the donor and acceptor are separated by an RERS as in SEQ ID NO: 16 and SEQ ID NO: 17. Strand displacement by the polymerase converts the reporter to double-stranded form by synthesis of a complementary strand. The RERS also becomes double-stranded and cleavable by the restriction endonuclease.

It will be apparent that, in addition to SDA, the detector oligonucleotides of the present invention may be adapted for use in the detection of amplicons in other primer extension amplification methods (e.g., PCR, 3SR, TAS or NASBA). For example, but not by way of limitation, the methods of the present invention may be adapted for use in PCR by using PCR amplification primers and a strand displacing DNA polymerase which lacks 5′→3′ exonuclease activity (e.g., Sequencing Grade Taq from Promega or exo-Vent or exo-Deep Vent from New England BioLabs) in the PCR. The signal primers hybridize to the target at least partially downstream from the PCR amplification primers, are displaced, and are rendered double-stranded essentially as described for SDA. In PCR, any RERS may optionally be selected for use in the reporter oligonucleotide, as there are typically no modified deoxynucleoside triphosphates present that might induce nicking rather than cleavage of the RERS. As thermocycling is a feature of amplification by PCR, the restriction endonuclease is preferably added at low temperature after the final cycle of primer annealing and extension for end-point detection of amplification. A thermophilic restriction endonuclease that remains active through the high temperature phases of the PCR reaction could, however, be present during amplification to provide a real-time assay. As in SDA systems, separation of the dye pair reduces fluorescence quenching, with a change in a fluorescence parameter such as intensity serving as an indication of target amplification.

Because most patients show few symptoms of CMV infection, quantification of the virus is an important consideration for diagnosis and treatment. The methods of the present invention are well-suited for this analysis. For example, the change in fluorescence resulting from unfolding, linearization and/or cleavage of the reporter oligonucleotides may be detected at a selected endpoint in the reaction. Because linearized secondary structures and/or cleaved reporter molecules are produced concurrently with hybridization or primer extension, the change in fluorescence may also be monitored as the reaction is occurring, i.e., in “real-time.” This homogeneous, real-time assay format may be used to provide semiquantitative or quantitative information about the initial amount of target present. For example, but not by way of limitation, the rate at which fluorescence intensity changes during the unfolding or linearizing reaction (either as part of target amplification or in non-amplification detection methods) is an indication of initial target levels. As a result, when more initial copies of the target sequence are present, donor fluorescence more rapidly reaches a selected threshold value (i.e., shorter time to positivity). The decrease in acceptor fluorescence similarly exhibits a shorter time to positivity, detected as the time required to reach a selected minimum value. In addition, the rate of change in fluorescence parameters during the course of the reaction is more rapid in samples containing higher initial amounts of target than in samples containing lower initial amounts of target (i.e., increased slope of the fluorescence curve). These or other measurements as are known in the art (e.g., U.S. Pat. Nos. 5,928,907 and 6,216,049, both of which are incorporated herein by reference in their entirety) may be made as an indication of the presence of target or as an indication of target amplification. The initial amount of target is typically determined by comparison of the experimental results to results for known amounts of target.

Assays for the presence of a selected target sequence according to the methods of the invention may be performed in solution or on a solid phase. Real-time or endpoint homogeneous assays in which the reporter oligonucleotide functions as a primer are typically performed in solution. Hybridization assays using the reporter oligonucleotides of the invention may also be performed in solution (e.g., as homogeneous real-time assays) but are also particularly well-suited to solid-phase assays for real-time or endpoint detection of target. In a solid-phase assay, reporter oligonucleotides may be immobilized on the solid phase (e.g., beads, membranes or the reaction vessel) via internal or terminal labels using methods known in the art. For example, but not by way of limitation, a biotin-labeled reporter oligonucleotide may be immobilized on an avidin-modified solid phase where it will produce a change in fluorescence when exposed to the target under appropriate hybridization conditions. Capture of the target in this manner facilitates separation of the target from the sample and allows removal of substances in the sample that may interfere with detection of the signal or other aspects of the assay. An example of a solid-phase system that can be used is an array format known in the art.

The following illustrative non-limiting Example illustrates specific embodiments of the invention described herein. As would be apparent to skilled artisans, various changes and modifications are possible, and are contemplated within the scope of the invention described.

### EXAMPLE

Use of primers and probes of the invention may be exemplified using an SDA reaction to detect CMV. For such a reaction, one “upstream” amplification primer is selected from SEQ ID NOs:1 and 2 and one “downstream” primer is selected from SEQ ID NOs:3-5. A signal primer is also selected from SEQ ID NOs:12-15, as well as a reporter probe such as SEQ ID NOs.:16 and 17, which are labeled with a donor/quencher dye pair as is known in the art for detection of target amplification. Rhodamine and fluorescein are preferred donor dyes for this purpose, while dabcyl is a preferred quencher. Finally, SEQ ID NOs: 9, 10, or 11 serves as the “upstream” bumper primer and SEQ ID NOs.:6, 7, or 8 serves as the “downstream” bumper primer. SDA is preferably performed at about 52° C. as described in U.S. Pat. No. 5,648,211 using the selected reporter to provide detection of the target during amplification as described in U.S. Pat. Nos. 5,919,630, 5,928,869 and 5,958,700.

Donor fluorescence is monitored during the amplification reaction. In the presence of CMV target nucleic acids, donor fluorescence will increase as the donor and quencher are separated following cutting at the RERS. In the absence of target, fluorescence will remain consistently low throughout the reaction. An increase in fluorescence or a failure of fluorescence to change substantially indicate the presence or absence of CMV target, respectively. Typically, the generation of relatively higher amount of fluorescence indicates a higher initial level of target.

What is claimed is:

1. An oligonucleotide having a sequence consisting essentially of a target binding sequence of any one of SEQ ID NOs:1 through 5.

2. The oligonucleotide of claim 1 which consists essentially of the target binding sequence of SEQ ID NOs:2 or 5.

3. The oligonucleotide of claim 1, further comprising a hairpin, G-quartet, restriction site or a sequence which hybridizes to a reporter probe.

4. The oligonucleotide of claim 1 which is labeled with a detectable label.

5. The oligonucleotide of claim 4 wherein the label is fluorescent.

6. The oligonucleotide of claim 1, further comprising a sequence required for an amplification or detection reaction.

7. The oligonucleotide of claim 6, wherein the sequence required for an amplification or detection reaction is a restriction endonuclease recognition site or a DNA polymerase promoter.

8. A kit for an amplification or detection reaction comprising an oligonucleotide having a sequence consisting essentially of the target binding sequence of any one of SEQ ID NOs:1 through 5.

9. The kit of claim 8, further comprising one or more bumper primers.

10. The kit of claim 9, wherein the one or more bumper primers consist essentially of SEQ ID NOs:6, 7, 8, 9, 10 or 11.

11. The kit of claim 8, further comprising a signal primer.

12. The kit of claim 8, further comprising a signal primer and a reporter probe, the signal primer consisting essentially of the target binding sequence of SEQ ID NO:12, 13, 14, or 15 and the reporter probe consisting essentially of the target binding sequence of SEQ ID NO:16 or 17.

13. The kit of claim 12, the signal primer consisting essentially of the target binding sequence of SEQ ID NO:14 and the reporter probe consisting essentially of the target binding sequence of SEQ ID NO:16.

14. A method for detecting the presence or absence of Cytomegalovirus (CMV) in a sample comprising:
(a) hybridizing a first primer having a sequence consisting essentially of the target binding sequence of any one of SEQ ID NOs: 1 through 5 to a target sequence and;
(b) detecting the hybridized target sequence.

15. The method of claim 14, further comprising a second primer having a sequence consisting essentially of the target binding sequence of any one of SEQ ID NOs: 1 through 5.

16. The method of claim 15, wherein the first primer consists essentially of the target binding sequence of SEQ ID NO:2 and the second primer consists essentially of the target binding sequence of SEQ ID NO:5.

17. The method of claim 14, wherein an amplification or detection reaction is used to detect the hybridized target sequence.

18. The method of claim 14, wherein said amplification or detection reaction is selected from the group consisting of Strand Displacement Amplification (SDA), polymerase chain reaction (PCR), transcription mediated amplification (TMA), self sustained sequence replication (SSR), rolling circle amplification or nucleic acid sequence based amplification (NASBA).

19. The method of claim 14, further comprising:
(a) combining the sample with a known concentration of CMV internal control nucleic acid;
(b) amplifying the target sequence and internal control nucleic acid in an amplification reaction;
(c) detecting the amplified target sequence and internal control nucleic acid; and
(d) analyzing the relative amounts of amplified target sequence and internal control nucleic acid.

20. The method of claim 14, wherein the first amplification primer further comprises a hairpin, G-quartet, restriction site or a sequence which hybridizes to a reporter probe.

21. The method of claim 14 wherein the first primer further comprises a restriction endonuclease recognition site or a DNA polymerase promoter.

22. An oligonucleotide having a sequence consisting essentially of SEQ ID NOs:1 through 5.

23. The oligonucleotide of claim 22 which consists essentially of SEQ ID NOs:2 or 5.

24. The oligonucleotide of claim 22, further comprising a hairpin, G-quartet, restriction site or a sequence which hybridizes to a reporter probe.

25. The oligonucleotide of claim 22 which is labeled with a detectable label.

26. The oligonucleotide of claim 25 wherein the label is fluorescent.

27. The oligonucleotide of claim 22, further comprising a sequence required for an amplification or detection reaction.

28. The oligonucleotide of claim 27, wherein the sequence required for an amplification or detection reaction is a restriction endonuclease recognition site or a DNA polymerase promoter.

29. A kit for an amplification or detection reaction comprising an oligonucleotide having a sequence consisting essentially of SEQ ID NOs:1 through 5.

30. The kit of claim 29, further comprising one or more bumper primers.

31. The kit of claim 29, wherein the one or more bumper primers consist essentially of SEQ ID NOs:6, 7, 8, 9, 10 or 11.

32. The kit of claim 29, further comprising a signal primer.

33. The kit of claim 29, further comprising a signal primer and a reporter probe, the signal primer consisting essentially of SEQ ID NO:12, 13, 14, or 15 and the reporter probe consisting essentially of SEQ ID NO:16 or 17.

34. The kit of claim 33, the signal primer consisting essentially of SEQ ID NO:14 and the reporter probe consisting essentially of SEQ ID NO:16.

35. A method for detecting the presence or absence of Cytomegalovirus (CMV) in a sample comprising:
(a) hybridizing a first primer having a sequence consisting essentially of SEQ ID NOs: 1 through 5 to a target sequence and;
(b) detecting the hybridized target sequence.

36. The method of claim 35, further comprising a second primer having a sequence consisting essentially of SEQ ID NOs: 1 through 5.

37. The method of claim 36, wherein the first primer consists essentially of the SEQ ID NO:2 and the second primer consists essentially of SEQ ID NO:5.

38. The method of claim 35, wherein an amplification or detection reaction is used to detect the hybridized target sequence.

39. The method of claim 35, wherein said amplification or detection reaction is selected from the group consisting of Strand Displacement Amplification (SDA), polymerase chain reaction (PCR), transcription mediated amplification (TMA), self sustained sequence replication (SSR), rolling circle amplification or nucleic acid sequence based amplification (NASBA).

40. The method of claim 35, further comprising:
(a) combining the sample with a known concentration of CMV internal control nucleic acid;
(b) amplifying the target sequence and internal control nucleic acid in an amplification reaction;
(c) detecting the amplified target sequence and internal control nucleic acid; and
(d) analyzing the relative amounts of amplified target sequence and internal control nucleic acid.

41. The method of claim 35, wherein the first amplification primer further comprises a hairpin, G-quartet, restriction site or a sequence which hybridizes to a reporter probe.

42. The method of claim 35 wherein the first primer further comprises a restriction endonuclease recognition site or a DNA polymerase promoter.


SEQUENCE LISTING (17 sequences)

SEQ ID NO: 1 - DNA, 39 bp, Artificial Sequence
  Description of Artificial Sequence Synthetic primer
cgattccgctccagacttctcgggcgcgtcaagaactct

SEQ ID NO: 2 - DNA, 41 bp, Artificial Sequence
  Description of Artificial Sequence Synthetic primer
cgattccgctccagacttctcgggcgcgtcaagaactctac

SEQ ID NO: 3 - DNA, 38 bp, Artificial Sequence
  Description of Artificial Sequence Synthetic primer
accgcatcgaatgcatgtctcgggtctccgtcgtatgt

SEQ ID NO: 4 - DNA, 39 bp, Artificial Sequence
  Description of Artificial Sequence Synthetic primer
accgcatcgaatgcatgtctcgggctctccgtcgtatgt

SEQ ID NO: 5 - DNA, 40 bp, Artificial Sequence
  Description of Artificial Sequence Synthetic primer
accgcatcgaatgcatgtctcgggtctctccgtcgtatgt

SEQ ID NO: 6 - DNA, 16 bp, Artificial Sequence
  Description of Artificial Sequence Synthetic primer
tttctttcagccttcg

SEQ ID NO: 7 - DNA, 16 bp, Artificial Sequence
  Description of Artificial Sequence Synthetic primer
ttttctttcagccttc

SEQ ID NO: 8 - DNA, 16 bp, Artificial Sequence
  Description of Artificial Sequence Synthetic primer
cttttctttcagcctt

SEQ ID NO: 9 - DNA, 15 bp, Artificial Sequence
  Description of Artificial Sequence Synthetic primer
tgaagatttcgcgtc

SEQ ID NO: 10 - DNA, 15 bp, Artificial Sequence
  Description of Artificial Sequence Synthetic primer
cgatgaagatttcgc

SEQ ID NO: 11 - DNA, 16 bp, Artificial Sequence
  Description of Artificial Sequence Synthetic primer
tacgatgaagatttcg

SEQ ID NO: 12 - DNA, 41 bp, Artificial Sequence
  Description of Artificial Sequence Synthetic primer
acgttagccaccatacggattcatgggcagcctcgtccact

SEQ ID NO: 13 - DNA, 42 bp, Artificial Sequence
  Description of Artificial Sequence Synthetic primer
acgttagccaccatacggattcatgggcagcctcgtccactc

SEQ ID NO: 14 - DNA, 43 bp, Artificial Sequence
  Description of Artificial Sequence Synthetic primer
acgttagccaccatacggattcatgggcagcctcgtccactcc

SEQ ID NO: 15 - DNA, 42 bp, Artificial Sequence
  Description of Artificial Sequence Synthetic primer
acgttagccaccatacggatcatggagtggacgaggctgccc

SEQ ID NO: 16 - DNA, 29 bp, Artificial Sequence
  Description of Artificial Sequence Synthetic probe
tccccgagtactgatccgcactaacgact

SEQ ID NO: 17 - DNA, 29 bp, Artificial Sequence
  Description of Artificial Sequence Synthetic probe
tccccgagtacgttagccaccatacttga